SEQUENCE LISTING (33 sequences)

SEQ ID NO: 1 - DNA, 19 bp, HIV-1
  misc_feature (1)..(19): 143 to 161 5′ LTR U3
gctagtaccagttgagcca

SEQ ID NO: 2 - DNA, 19 bp, HIV-1
  misc_feature (1)..(19): strain BaL, subtypes B, D, F, H, J, K (143 to 161 5′ LTR U3)
gctagtaccagttgagcca

SEQ ID NO: 3 - DNA, 19 bp, HIV-1
  misc_feature (1)..(19): subtype A (143 to 161 5′ LTR U3)
gctagtaccagttgatcca

SEQ ID NO: 4 - DNA, 19 bp, HIV-1
  misc_feature (1)..(19): subtype C (143 to 161 5′ LTR U3)
gctagtgccagttgaccca

SEQ ID NO: 5 - DNA, 19 bp, HIV-1
  misc_feature (1)..(19): subtype G (143 to 161 5′ LTR U3)
gctagtaccagtggaccca

SEQ ID NO: 6 - RNA, 19 nt, Artificial Sequence
  Synthetic  RNA duplex sense strand (143 to 161 5′ LTR U3)
gcuaguaccaguugagcca

SEQ ID NO: 7 - RNA, 19 nt, Artificial Sequence
  Synthetic  RNA duplex antisense strand (143 to 161 5′ LTR U3)
uggcucaacugguacuagc

SEQ ID NO: 8 - RNA, 57 nt, Artificial Sequence
  Synthetic  shRNA (143 to 161 5′ LTR U3)
gcuaguaccaguugagccacugugaagccacagauggguggcucaacugguacuagc

SEQ ID NO: 9 - DNA, 19 bp, HIV-1
  misc_feature (1)..(19): 136 to 154 5′ LTR U3, subtype A
gctacaagctagtaccagt

SEQ ID NO: 10 - DNA, 19 bp, HIV-1
  misc_feature (1)..(19): strain SF162, subtypes B, F, H, J, K (136 to 154 5′ LTR U3)
gcttcaagctagtaccagt

SEQ ID NO: 11 - DNA, 19 bp, HIV-1
  misc_feature (1)..(19): subtype C (136 to 154 5′ LTR U3)
gcttcaagctagtgccagt

SEQ ID NO: 12 - DNA, 19 bp, HIV-1
  misc_feature (1)..(19): subtype D (136 to 154 5′ LTR U3)
gcttcgagctagtaccagt

SEQ ID NO: 13 - DNA, 19 bp, HIV-1
  misc_feature (1)..(19): subtype G (136 to 154 5′ LTR U3)
gcttcaaactagtaccaat

SEQ ID NO: 14 - DNA, 19 bp, Artificial Sequence
  Synthetic  RNA duplex sense strand (136 to 154 5′ LTR U3)
gcuucaagcuaguaccagu

SEQ ID NO: 15 - RNA, 19 nt, Artificial Sequence
  Synthetic  RNA duplex antisense strand (136 to 154 5′ LTR U3)
acugguacuagcuugaagc

SEQ ID NO: 16 - DNA, 57 bp, Artificial Sequence
  Synthetic  shRNA (136 to 154 5′ LTR U3)
gcuucaagcuaguaccagucugugaagccacagaugggacugguacuagcuugaagc

SEQ ID NO: 17 - DNA, 19 bp, HIV-1
  misc_feature (1)..(19): 205 to 223 5′ LTR U3
acaccctatgagcctgcat

SEQ ID NO: 18 - DNA, 19 bp, HIV-1
  misc_feature (1)..(19): strain NL4-3, Subtype B, H, J, K (205 to 223 5′ LTR U3)
acaccctatgagcctgcat

SEQ ID NO: 19 - DNA, 19 bp, HIV-1
  misc_feature (1)..(19): subtype C and D (205 to 223 5′ LTR U3)
acaccctatgagccagcat

SEQ ID NO: 20 - DNA, 19 bp, HIV-1
  misc_feature (1)..(19): Subtype F (205 to 223 5′ LTR U3)
acaccccatgagccaacat

SEQ ID NO: 21 - DNA, 19 bp, HIV-1
  misc_feature (1)..(19): Subtype G (205 to 223 5′ LTR U3)
acaccccatctgccagcat

SEQ ID NO: 22 - DNA, 19 bp, Artificial Sequence
  Synthetic  RNA duplex sense strand (205 to 223 5′ LTR U3)
acacccuaugagccugcau

SEQ ID NO: 23 - RNA, 19 nt, Artificial Sequence
  Synthetic  RNA duplex antisense strand (205 to 223 5′ LTR U3)
augcaggcucauagggugu

SEQ ID NO: 24 - DNA, 57 bp, Artificial Sequence
  Synthetic  shRNA (205 to 223 5′ LTR U3)
acacccuaugagccugcaucugugaagccacagaugggaugcaggcucauagggugu

SEQ ID NO: 25 - DNA, 30 bp, Artificial Sequence
  Synthetic  HIV-gag sense primer
agtggggggacatcaagcagccatgcaaat

SEQ ID NO: 26 - DNA, 28 bp, Artificial Sequence
  Synthetic  HIV-gag antisense primer
tactagtagttcctgctatgtcacttcc

SEQ ID NO: 27 - DNA, 29 bp, Artificial Sequence
  Synthetic  HIV-gag detection probe
  misc_feature: 5′ FAM / 3′ TAMRA
atcaatgaggaagctgcagaatgggatag

SEQ ID NO: 28 - DNA, 25 bp, Artificial Sequence
  Synthetic  Beta-actin sense primer
tcacccacactgtgcccatctacga

SEQ ID NO: 29 - DNA, 25 bp, Artificial Sequence
  Synthetic  Beta-actin anti-sense primer
cagcggaaccgctcattgccaatgg

SEQ ID NO: 30 - DNA, 25 bp, Artificial Sequence
  Synthetic  Beta-actin detection probe
  misc_feature: 5′ FAM / 3′ TAMRA
atgccctcccccatgccatcctgcg

SEQ ID NO: 31 - DNA, 24 bp, Artificial Sequence
  Synthetic  Primer set of NUAf
ccaaagaagacaagatatccttga

SEQ ID NO: 32 - DNA, 24 bp, Artificial Sequence
  Synthetic  Primer set of Chips2r
gcagctgcttatatgcagcatctg

SEQ ID NO: 33 - DNA, 19 bp, HIV-1
  misc_feature: Strain SF162, subtype A (205 to 223 5′ LTR U3)
acaccctatatgccaacat

(19) United States
(12) Patent Application Publication
(10) Pub. No.: US 2017/0298357 A1
(43) Pub. Date: Oct. 19, 2017

(54) Treatment of HIV

(71) Applicants: Chantelle Lisa Evelyn AHLENSTIEL, Sydney, New South Wales (AU); Anthony Dominic KELLEHER, Sydney, New South Wales (AU); Maria Catalina MENDEZ, Sydney, New South Wales (AU); Kazuo SUZUKI, Sydney, New South Wales (AU); NEWSOUTH INNOVATIONS PTY LIMITED, SYDNEY (AU); ST VINCENT'S HOSPITAL SYDNEY LIMITED, SYDNEY (AU)

(72) Inventors: Chantelle Lisa Evelyn AHLENSTIEL, Sydney (AU); Anthony Dominic KELLEHER, Sydney (AU); Maria Catalina MENDEZ, Sydney (AU); Kazuo SUZUKI, Sydney (AU)

(21) Appl. No.: 15/507,308

(22) PCT Filed: Aug. 28, 2015
(86) PCT No.: PCT/AU2015/050507

§ 371 Date: Feb. 28, 2017

(30) Foreign Application Priority Data
Aug. 28, 2014 (AU) 2014903428

(51) Int. Cl. C12N 15/113

(52) U.S. Cl. CPC C12N 15/1132; C12N 2310/14

(57) ABSTRACT

Described herein are silencing nucleic acids, compositions comprising silencing nucleic acids, and methods of utilizing the silencing nucleic acids to to inhibit HIV replication in a cell infected with HIV. In some embodiments, the disclosed methods comprise contacting a cell or population of cells infected with HIV with at least one silencing nucleic acid that targets a specific sequence of the 5′ LTR of HIV.

### CROSS REFERENCE TO RELATED APPLICATIONS

The present application claims the benefit of the filing date of Australian Provisional Patent Application No. 2014903428 filed Aug. 28, 2014, the disclosure of which is hereby incorporated by reference herein in its entirety.

### FIELD

The invention relates to a method of inhibiting HIV replication, to a method of treating HIV infection in a subject in need thereof, to a method of preventing or reducing HIV infection in a subject, and to a nucleic acid which inhibits HIV replication.

### BACKGROUND

HIV is the causative agent of Acquired Immunodeficiency Syndrome (AIDS). Tens of millions of people are infected with HIV worldwide.

HIV belongs to the retroviridae family of viruses, and is an envelope virus whose genome consists of two single stranded RNA molecules (ssRNA). The primary target of HIV is CD4+ expressing cells, such as CD4+ T cells. Glycoprotein of the HIV virus interacts with the CD4 molecule of target cells and with chemokine co-receptors, CCR5 and CXCR4 on the surface of target cells. Following fusion and entry into the target cell, the nucleocapsid containing the viral genome dissociates, releasing the contents of the virus, including the ssRNA, into the cytoplasm. A reverse transcriptase (RT) enzyme of HIV synthesizes viral double stranded DNA (dsDNA) from the ssRNA genome. Following synthesis of the double stranded HIV DNA molecule, the HIV DNA is integrated into the host genome.

The integrated HIV DNA is flanked by identical 5′ and 3′ long terminal repeat sequences (LTR) from which HIV can initiate transcription of the integrated HIV genome. Transcription of the viral DNA requires host transcription factors, such as NF-κB, which are upregulated in activated T cells and act in concert with T the virally encoded transcriptional enhancer. As a consequence, viral transcription is most active in the T cell following activation of the T cell, such as during infection. Viral RNA resulting from transcription of the integrated HIV genome is subsequently translated and packaged into virus particles which then exit the cell to become infectious virus.

Therapy for HIV infection includes combination antiretroviral therapy (cART), which dramatically slows HIV progression. However, cART therapy can be compromised by drug resistant mutations, and has a range of serious side effects which appear cumulative. Further, interruption of cART therapy almost invariably leads to the re-emergence of detectable viral replication and the progression to AIDS and has been shown to be associated with an increased incidence of all causes of mortality and serious non AIDS events. While cART reduces the extent of proviral infection, its effect on this form of the virus is relatively limited. This residual provirus forms a viral reservoir, which resides in long-lived resting T cells and tissue-based macrophages.

The latent viral reservoir is difficult to manipulate. None of the currently available antiretroviral agents act directly on the reservoir, though effective use of cART in compliant individuals for 30-40 years may result in elimination of the reservoir. cART initiates a two phase decay of proviral DNA: a rapid first phase, reaching an inflection point at approximately 3 to 6 months after commencing cART and; a much slower second phase, likely due to the natural death of cells containing integrated provirus. Intensification of cART by increasing the number of drug classes in a regimen does not increase the rate of reservoir decay.

Early intervention with cART during the first 6 months of infection, when the resting T cells reservoir accumulates, appears to limit the reservoir size and reduces it approximately 10-fold compared to cART commenced during chronic infection. However, the reservoir is still established and the decay rates of the reservoir are no different between the two groups. Given these limitations, alternate means of manipulating the reservoir are required.

Latent HIV integrated virus has epigenetic modifications associated with the 5′LTR. The 5′LTR acts as the viral promoter and is the universal transcription controller for all HIV genes. These epigenetic modifications consist of deacetylation and methylation of a number of lysine residues within histone 3, which aggregate in one of the two nucleosomes (nuc) associated with the 5′LTR. These epigenetic marks are associated with chromatin compaction and lack of host transcription factor binding to specific motifs located within the 5′LTR. There has been substantial effort aimed at manipulating this state. Most have attempted to reactivate virus, particularly in memory T cells, via cell activation or altering viral epigenetic profiles. This “Kick and Kill” approach putatively impacts on the reservoir by causing viral reactivation, in the presence of cART, preventing ongoing infection. The reactivation ‘kick” aims to induce “killing” of cells either directly, or through the production of viral antigens making them targets for the immune system. Global T cell activation with, such as IL-2, IL-7 or OKT3 has been attempted without success.

### SUMMARY OF THE INVENTION

A first aspect provides a method of inhibiting HIV replication in a cell infected with HIV, comprising contacting the cell with an effective amount of at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. Numbering of the silencing nucleic acid is based on the 5′LTR U3 start site in the subtype B HXB2 strain (Accession no. K03455) (Wong-Staal, F. 1985, Nature 313:277-284). In some embodiments, the method comprises administering at least two of the silencing nucleic acids.

A second aspect provides a method of treating HIV infection in a subject, comprising administering to the subject an effective amount of at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the method comprises administering at least two of the silencing nucleic acids.

An alternative second aspect provides the use of at least one silencing nucleic acid in the manufacture of a medicament for treating an HIV infection in a subject or to inhibit HIV gene transcription for use in treating an HIV infection in a subject, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1.

A third aspect provides a method of preventing or reducing HIV infection in a subject, comprising administering to the subject an effective amount of at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the method comprises administering at least two of the silencing nucleic acids.

An alternative third aspect provides the use of at least one silencing nucleic acid in the manufacture of a medicament for preventing or reducing HIV infection in a subject suffering from an HIV infection or to inhibit HIV gene transcription for use in preventing or reducing HIV infection in a subject suffering from an HIV infection, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1.

A fourth aspect provides a method of preventing or reducing HIV infection in a subject suffering from an HIV infection, comprising administering to the subject an effective amount of at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the method comprises administering at least two of the silencing nucleic acids.

An alternative fourth aspect provides the use of at least one silencing nucleic acid in the manufacture of a medicament for preventing or reducing HIV infection in a subject suffering from an HIV infection or to inhibit HIV gene transcription for use in preventing or reducing HIV infection in a subject suffering from an HIV infection, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1.

A fifth aspect provides a method of preventing or reducing a productive HIV infection in a subject not suffering from an HIV infection, comprising administering to the subject an effective amount of at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the method comprises administering at least two of the silencing nucleic acids.

An alternative fifth aspect provides the use of at least one silencing nucleic acid in the manufacture of a medicament for preventing or reducing a productive HIV infection in a subject not suffering from an HIV infection or to inhibit HIV gene transcription for use in preventing or reducing a productive HIV infection in a subject not suffering from an HIV infection, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1.

A sixth aspect provides at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1.

An alternate sixth aspect provides a silencing nucleic acid to inhibit HIV gene transcription, where the silencing nucleic acid targets a sequence from about position 143 to about position 161 of the 5′ LTR of HIV-1. In some embodiments, the silencing nucleic acid targets a sequence having at least 95% identity with the sequence of SEQ ID NO: 1. In some embodiments, the silencing nucleic acid targets the sequence of SEQ ID NO: 1. In some embodiments, the silencing nucleic acid is an RNA duplex comprising a sense and an antisense strand, wherein the sense strand comprises a sequence having at least 95% identity to the sequence of SEQ ID NO: 6, and the antisense strand comprises a sequence that is complementary to the sense strand. In some embodiments, the RNA duplex is an siRNA. In some embodiments the siRNA comprises a sense strand having the sequence of SEQ ID NO: 6, and an antisense strain having the sequence of SEQ ID NO: 7. In some embodiments, the RNA duplex is an shRNA. In some embodiments the shRNA comprises the sequence of SEQ ID NO: 8.

Another alternate sixth aspect provides a silencing nucleic acid to inhibit HIV gene transcription, where the silencing nucleic acid targets a sequence from about position 136 to about position 154 of the 5′ LTR of HIV-1. In some embodiments, the silencing nucleic acid targets a sequence having at least 95% identity with the sequence of SEQ ID NO: 9. In some embodiments, the silencing nucleic acid targets the sequence of SEQ ID NO: 9. In some embodiments, the silencing nucleic acid is an RNA duplex comprising a sense and an antisense strand, wherein the sense strand comprises a sequence having at least 95% identity to the sequence of SEQ ID NO: 14, and the antisense strand comprises a sequence that is complementary to the sense strand. In some embodiments, the RNA duplex is an siRNA. In some embodiments, the siRNA comprises a sense strand having the sequence of SEQ ID NO: 14, and an antisense strain having the sequence of SEQ ID NO: 15. In some embodiments, the RNA duplex is an shRNA. In some embodiments, the shRNA comprises the sequence of SEQ ID NO: 16.

A further alternate sixth aspect provides a silencing nucleic acid to inhibit HIV gene transcription, where the silencing nucleic acid targets a sequence from about position 205 to about position 223 of the 5′ LTR of HIV-1. In some embodiments, the silencing nucleic acid targets a sequence having at least 95% identity with the sequence of SEQ ID NO: 17. In some embodiments, the silencing nucleic acid targets the sequence of SEQ ID NO: 17. In some embodiments, the silencing nucleic acid is an RNA duplex comprising a sense and an antisense strand, wherein the sense strand comprises a sequence having at least 95% identity to SEQ ID NO: 22, and the antisense strand comprises a sequence that is complementary to the sense strand. In some embodiments, the RNA duplex is an siRNA. In some embodiments, the siRNA comprises a sense strand having the sequence of SEQ ID NO: 22, and an antisense strain having the sequence of SEQ ID NO: 23. In some embodiments, the RNA duplex is an shRNA. In some embodiments, the shRNA comprises the sequence of SEQ ID NO: 24.

A seventh aspect provides a composition, formulation, conjugate or construct comprising at least one silencing nucleic acid, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the composition further comprises a pharmaceutically acceptable carrier. In some embodiments, a composition or formulation further comprises a pharmaceutically acceptable carrier. In some embodiments, the composition or formulation is formulated as an emulsion. In some embodiments, the composition or formulation is formulated with micelles or nanoparticles. In some embodiments, the silencing nucleic acids in any composition, formulation or construct are encapsulated within a polymer (e.g. a bioresorbable polymer). In some embodiments, the silencing nucleic acids are encapsulated within liposomes. In some embodiments, the silencing nucleic acids are encapsulated within minicells.

An eighth aspect provides a kit comprising at least one silencing nucleic acid, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the kit comprises two of the silencing nucleic acids. In some embodiments, the kit comprises three of the silencing nucleic acids.

A ninth aspect provides a cell comprising at least one silencing nucleic acid to inhibit HIV gene transcription, wherein the at least one silencing nucleic acid is selected from the group consisting of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and (iii) a silencing nucleic acid which targets a sequence from about position 205 to about position 223 of the 5′ LTR of HIV-1. In some embodiments, the cell is prepared by transferring the silencing nucleic acids into the cell. In some embodiments, the cell comprises at least two of the silencing nucleic acids.

A tenth aspect provides a composition comprising at least two of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; and (iii) an siRNA comprising a sense strand having the sequence of SEQ ID NO: 22, and an antisense strand having the sequence of SEQ ID NO: 23. In some embodiments, the composition comprises (i) an siRNA comprising a sense strand having the sequence of SEQ ID NO: 6, and an antisense strand having the sequence of SEQ ID NO: 7; and (ii) an siRNA comprising a sense strand having the sequence of SEQ ID NO: 14, and an antisense strand having the sequence of SEQ ID NO: 15. In some embodiments, the composition comprises (i) an siRNA comprising a sense strand having the sequence of SEQ ID NO: 6, and an antisense strand having the sequence of SEQ ID NO: 7; and (ii) an siRNA comprising a sense strand having the sequence of SEQ ID NO: 22, and an antisense strand having the sequence of SEQ ID NO: 23. In some embodiments, the composition comprises (i) an siRNA comprising a sense strand having the sequence of SEQ ID NO: 14, and an antisense strand having the sequence of SEQ ID NO: 15; and (ii) an siRNA comprising a sense strand having the sequence of SEQ ID NO: 22, and an antisense strand having the sequence of SEQ ID NO:23. In some embodiments, the composition comprises all three siRNAs. In some embodiments, the compositions consist essentially of at least two of the siRNAs. In some embodiments, consist of at least two of the siRNAs.

An eleventh aspect provides a composition comprising at least two of (i) an shRNA having the sequence of SEQ ID NO: 8; (ii) an shRNA having the sequence of SEQ ID NO: 16; and (iii) an shRNA having the sequence of SEQ ID NO: 24. In some embodiments, the composition comprises (i) an shRNA having the sequence of SEQ ID NO: 8; and (ii) an shRNA having the sequence of SEQ ID NO: 16. In some embodiments, the composition comprises (i) an shRNA having the sequence of SEQ ID NO: 8; and (ii) an shRNA having the sequence of SEQ ID NO: 24. In some embodiments, the composition comprises (i) an shRNA having the sequence of SEQ ID NO: 16; and (ii) an shRNA having the sequence of SEQ ID NO: 24. In some embodiments, the composition comprises all three shRNAs. In some embodiments, the compositions consist essentially of at least two of the shRNAs. In some embodiments, consist of at least two of the shRNAs.

The inventors have surprisingly and unexpectedly discovered that a silencing nucleic acid which targets the 5′ LTR of HIV-1 in the region from position 143 to position 161 effectively inhibits transcription of the HIV genome. Similarly, the inventors have also surprisingly and unexpectedly discovered that a silencing nucleic acid which targets the 5′ LTR of HIV1 in the region from position 136 to position 154 effectively inhibits transcription of the HIV genome. In addition, the inventors have also surprisingly and unexpectedly discovered that a silencing nucleic acid which targets the 5′ LTR of HIV-1 in the region from position 205 to position 233 effectively inhibits transcription of the HIV genome.

Moreover, Applicants believe that HIV latency can be induced and maintained by delivery and expression of single or multiple silencing nucleic acids targeting the viral promoter that mediate transcriptional gene silencing and can do so with sufficient viral coverage and resistance to viral reactivation by inflammatory and/or immunological stimuli, to allow the viral reservoir to be stabilized for prolonged periods. Without wishing to be bound by any particular theory, Applicants believe that this approach will provide increased efficacy and long-term control of HIV infection and control or reduction in the viral reservoir in the absence of combined antiretroviral therapy.

Without wishing to be bound by any particular theory, Applicants believe that short hairpin RNAs, in association with Ago1, induce biochemical and structural changes in the architecture of the chromatin associated with the 5′LTR, inducing deacetylation and methylation of lysines K9 and K27 of histone 3, recruiting histone methytransferases, Enhancer of Zeste and recruitment of HDAC. These changes result in the shifting of the position of nucleosome 1 and incorporation of the transcription start site into nucleosome bound DNA. These modifications are believed to be very similar to the epigenetic changes described in latent HIV infection (see Siliciano, R. F. & Greene, W. C. HIV latency. Cold Spring Harb Perspect Med 1, a007096 (2011). It is believed that the compositions and/or constructs described herein can induce and maintain epigenetic silencing in T cell lines, primary CD4+ T cells, macrophage cell lines and monocyte-derived macrophage and dendritic cells. The effect is believed to be highly specific and Applicants have been unable to demonstrate any significant off-target effects in terms of alterations of CD4+ T cell or CD34+ HSC differentiation, expression of cell surface markers, proliferative ability or production of type 1 interferons.

### BRIEF DESCRIPTION OF THE FIGURES

FIG. 1 is a graph showing the results of a flow cytometry analysis of pseudotyped HIV-GFP expression in 293T cells transfected with novel candidate short interfering siRNAs targeting the HIV-1 5′LTR region. Pseudovirus-infected 293T cells were transfected with a panel of siRNAs targeting the HIV 5′LTR sequence. GFP expression was measured by flow cytometry 48 hours post-transfection of siRNA. Data are shown as mean±SD from three independent experiments. ** p=≦0.008.

FIG. 2A is a diagram showing the region within HIV-1 5′LTR targeted by the 143, 136, 205 and PromA siRNAs.

FIG. 2B is a graph showing the effect of siRNAs on the time course of HIV-1 BaL reverse transcriptase production in MAGIC-5 cells.

FIG. 2C is a graph showing the effect of siRNAs on the time course of HIV-1 SF162 reverse transcriptase production in HeLa T4+ cells.

FIG. 3 is an alignment of siRNA 143, 136, 205 and PromA with relatively conserved regions across HIV-1 A to G and U subtypes when aligned against subtype B strain HXB2 as shown. Identity and gaps are indicated by dots and dashes, respectively. Sequences were obtained from the Los Alamos National Laboratory-HIV Sequence Consortium 2013 database (HIV Sequence Compendium 2013 Foley B, Leitner T, Apetrei C, Hahn B, Mizrachi I, Mullins J, Rambaut A, Wolinsky S, and Korber B, Eds. Published by Theoretical Biology and Biophysics Group, Los Alamos National Laboratory, NM, LA-UR 13-26007).

FIG. 4A is a map of a construct (CMV-3LTR1-4) used for expression of the HIV-1 3′LTR under control of the immediate early CMV promoter, with the location of sequences targeted by the selected siRNAs (143/143T; and PromA) and positive control siRNAs, PolyA and Nef366. The position of PCR primers used for the detection of HIV-LTR mRNA is indicated with arrows.

FIG. 4B is a graph showing relative amounts of mRNA reduction through the PTGS pathway following transfection of HeLa cells, stably expressing CMV-3LTR1-4, with the siRNA as indicated. Real time PCR data are shown as a relative reduction in HIV-mRNA levels normalized to the mock transfection control. Data shown are from three independent experiments (mean±SEM). * p=0.028, ** p=0.009.

FIG. 5A is a graph showing suppression of HIV-1 following HIV infection and then transfection of siRNAs as indicated for 6 days, then analysis of intracellular viral RNA levels by RT-PCR. Real time PCR data are shown as a relative reduction in HIV-gag mRNA levels normalized GAPDH. Data shown are from three independent experiments (mean±SEM). ****p=≦0.0001. All statistical analyses were performed using a Mann-Whitney test comparing the siRNA to the mock control.

FIGS. 5B, 5C, 5D, and 5E consist of graphs showing drug induced reactivation of HIV transcription observed in HIV cultures suppressed by siRNAs as indicated, following HIV infection for 6 days and then transfection of siRNAs as indicated, treatment with TSA (50 nM), SAHA (2.5 μM), and TNF-α (10 ng/mL), or a combination of SAHA (2.5 μM) and TNF-α (10 ng/mL), then analysis of intracellular viral RNA levels by RT-PCR. Real time PCR data are shown as a relative reduction in HIV-gag mRNA levels normalized to the HIV+ mock transfection control. Data shown are from three independent experiments (mean±SEM). *p=≦0.03, **p=≦0.002, ***p=≦0.0002, ****p=≦0.0001. All statistical analyses were performed using a Mann-Whitney test comparing the siRNA alone control with the drug activation cultures.

FIGS. 6A, 6B, 6C, 6D, 6E, 6F, and 6G provide a series of graphs showing the levels of enrichment of the heterochromatin markers H3K27me3, H3K9me3 and Ago1 and reduction of heterochromatin mark H3K9Ac in siRNA 143, 143T-, PromA-, 136T- and 205S-transfected cultures compared to mock-transfected cultures. Data shown are from three independent experiments (mean±SEM). *p=≦0.03, **** p=≦0.0001.

FIG. 7A is a graph illustrating that J-Lat 9.2 cells transduced with combined short hairpin (sh)143 and PromA are able to suppress HIV replication as potently as single sh143 or shPromA transduced J-Lat 9.2 cells.

FIG. 7B is a graph showing that J-Lat 9.2 cells transduced with combined sh143 and shPromA and individual sh143 or shPromA are less susceptible to reactivation by combined TNF/SAHA treatments, particularly at physiological drug concentrations, compared to control cells, as shown by GFP expression, which increases upon reactivation.

### DETAILED DESCRIPTION OF THE INVENTION

Described herein are silencing nucleic acids, compositions comprising silencing nucleic acids, and methods of utilizing the silencing nucleic acids to to inhibit HIV replication in a cell infected with HIV. In some embodiments, the disclosed methods comprise contacting a cell or population of cells infected with HIV with at least one silencing nucleic acid that targets a specific sequence of the 5′ LTR of HIV. It is believed that the silencing nucleic acids that target the 5′ LTR sequences described herein are effective at inhibiting transcription of the HIV genome, and therefore represent a method by which HIV replication can be inhibited.

The inhibition of HIV replication may be by any mechanism that inhibits HIV replication. In some embodiments, the inhibition of HIV replication may be by transcriptional gene silencing (TGS). Without wishing to be bound by any particular theory, it is believed that TGS prevents transcription of the HIV genes and therefore prevents production of mRNA from the integrated HIV genome.

As used herein, a “cell infected with HIV” refers to a cell in which the HIV genome has integrated into the cell genome, and includes cells producing HIV virus, and cells latently infected with HIV. As used herein, a cell latently infected with HIV is a cell in which the HIV genome is integrated into the host cell genome, but which is transcriptionally inactive but capable of reactivation to a transcriptionally active state.

As used herein, “contacting the cell” refers to bringing a composition, formulation, or agent into contact with a cell in a manner which produces an effective result (i.e. reducing, mitigating, or eliminating HIV infection, transcription, or replication). Contacting the cell also includes introducing a composition, formulation, or agent into a cell.

As used herein, the phrases “inhibiting HIV transcription” or “inhibiting HIV replication” refers to reducing or preventing transcription of one or more HIV genes to the extent that infectious HIV virus particle formation in the cell is reduced, mitigated, or eliminated.

As used herein, the term “HIV” includes not only HIV-1, but also the various strains of HIV-1 (e.g. strain BaL or strain SF162) and the various subtypes of HIV-1 (e.g. subtypes A, B, C, D, F, G H, J, and K).

As used herein, “polynucleotide” refers to single or double stranded DNA, RNA, or modified versions thereof including peptide nucleic acid and locked nucleic acid (LNA).

As used herein, a “silencing nucleic acid” refers to any polynucleotide which is capable of interacting with a specific sequence to inhibit gene expression. Examples of silencing nucleic acids include RNA duplexes (e.g. siRNA, shRNA), LNAs, antisense RNA, DNA polynucleotides which encode sense and/or antisense sequences of the siRNA or shRNA, DNAzymse, or ribozymes. The inhibition of gene expression need not necessarily be gene expression from a specific enumerated sequence, and may be, for example, gene expression from a sequence controlled by that specific sequence.

In some embodiments, the method comprises contacting a cell or population of cells infected with HIV with one or more of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and/or (iii) a silencing nucleic acid which targets a sequence from about position 205 to about position 223 of the 5′ LTR of HIV-1. Without wishing to be bound by any particular theory, it is believed that the above-identified silencing nucleic acids are effective at inhibiting transcription of the HIV genome, and therefore represent a method by which HIV replication can be inhibited.

In some embodiments, the silencing nucleic acid targets a sequence having at least 95% identity with a target sequence from about position 143 to about position 161 of the 5′ LTR of HIV-1. In some embodiments, the silencing nucleic acid targets a sequence having at least 95% identity with that of SEQ ID NO: 1. In other embodiments, the silencing nucleic acid targets a sequence having at least 97% identity with that of SEQ ID NO: 1. In further embodiments, the silencing nucleic acid targets a sequence having at least 99% identity with that of SEQ ID NO: 1. In yet further embodiments, the silencing nucleic acid targets a sequence having about 100% identity with that of SEQ ID NO: 1.

In some embodiments, the silencing nucleic acid targets a sequence having at least 95% identity with a target sequence from about position 136 to about position 154 of the 5′ LTR of HIV-1. In some embodiments, the silencing nucleic acid targets a sequence having at least 95% identity with that of SEQ ID NO: 9. In yet other embodiments, the silencing nucleic acid targets a sequence having at least 97% identity with that of SEQ ID NO: 9. In further embodiments, the silencing nucleic acid targets a sequence having at least 99% identity with that of SEQ ID NO: 9. In yet further embodiments, the silencing nucleic acid targets a sequence having about 100% identity with that of SEQ ID NO: 9.

In some embodiments, the silencing nucleic acid targets a sequence having at least 95% identity with a target sequence from about position 205 to about position 223 of the 5′ LTR of HIV-1. In some embodiments, the silencing nucleic acid targets a sequence having at least 95% identity with that of SEQ ID NO: 17. In other embodiments, the silencing nucleic acid targets a sequence having at least 97% identity with that of SEQ ID NO: 17. In further embodiments, the silencing nucleic acid targets a sequence having at least 99% identity with that of SEQ ID NO: 17. In yet further embodiments, the silencing nucleic acid targets a sequence having about 100% identity with that of SEQ ID NO: 17.

As will be appreciated by those skilled in the art, there may be variation in the sequence of the 5′ LTR region between strains or subtypes of HIV-1, and thus there may be some variation in the target sequence of HIV-1. In some embodiments, the silencing nucleic acid targets one or more sequences selected from SEQ ID NO: 2, SEQ ID NO: 3, SEQ ID NO: 4, or SEQ ID NO: 5, or a sequence having at least 95% identity to one of SEQ ID NO: 2, SEQ ID NO: 3, SEQ ID NO: 4, or SEQ ID NO: 5. In other embodiments, the silencing nucleic acid targets one or more sequences selected from SEQ ID NO: 10, SEQ ID NO: 11, SEQ ID NO: 12, or SEQ ID NO: 13, or a sequence having at least 95% identity to one of SEQ ID NO: 10, SEQ ID NO: 11, SEQ ID NO: 12, or SEQ ID NO: 13. In yet other embodiments, the silencing nucleic acid targets one or more sequences selected from SEQ ID NO: 18, SEQ ID NO: 19, SEQ ID NO: 20, SEQ ID NO: 21, or SEQ ID NO 33, or a sequence having at least 95% identity to one of SEQ ID NO: 18, SEQ ID NO: 19, SEQ ID NO: 20, SEQ ID NO: 21, or SEQ ID NO 33.

In some embodiments, the silencing nucleic acid which targets the sequence from about position 143 to about position 161 of the 5′ LTR of HIV-1 is an RNA duplex comprising a sense strand and an antisense strand, wherein the sense strand comprises a sequence having at least 95% identity to that of SEQ ID NO: 6. In other embodiments, the silencing nucleic acid is an RNA duplex comprising a sense strand and an antisense strand, wherein the sense strand comprises a sequence having at least 97% identity to that of SEQ ID NO: 6. In further embodiments, the silencing nucleic acid is an RNA duplex comprising a sense strand and an antisense strand, wherein the sense strand comprises a sequence having at least 99% identity to that of SEQ ID NO: 6. In yet further embodiments, the silencing nucleic acid is an RNA duplex comprising a sense strand and an antisense strand, wherein the sense strand comprises a sequence having about 100% identity to that of SEQ ID NO: 6.

In some embodiments, the silencing nucleic acid which targets the sequence from about position 136 to about position 154 of the 5′ LTR of HIV-1 is an RNA duplex comprising a sense strand and an antisense strand, wherein the sense strand comprises a sequence having at least 95% identity to that of SEQ ID NO: 14. In other embodiments, the silencing nucleic acid is an RNA duplex comprising a sense strand and an antisense strand, wherein the sense strand comprises sequence having at least 97% identity to that of SEQ ID NO: 14. In further embodiments, the silencing nucleic acid is an RNA duplex comprising a sense strand and an antisense strand, wherein the sense strand comprises a sequence having at least 99% identity to that of SEQ ID NO: 14. In yet further embodiments, the silencing nucleic acid is an RNA duplex comprising a sense strand and an antisense strand, wherein the sense strand comprises a sequence having about 100% identity to that of SEQ ID NO: 14.

In some embodiments, the silencing nucleic acid which targets the sequence from about position 205 to about position 223 of the 5′ LTR of HIV-1 is an RNA duplex comprising a sense strand and an antisense strand, wherein the sense strand comprises a sequence having at least 95% identity to that of SEQ ID NO: 22. In other embodiments, the silencing nucleic acid is an RNA duplex comprising a sense strand and an antisense strand, wherein the sense strand comprises sequence having at least 97% identity to that of SEQ ID NO: 22. In further embodiments, the silencing nucleic acid is an RNA duplex comprising a sense strand and an antisense strand, wherein the sense strand comprises a sequence having at least 99% identity to that of SEQ ID NO: 22. In yet further embodiments, the silencing nucleic acid is an RNA duplex comprising a sense strand and an antisense strand, wherein the sense strand comprises a sequence having about 100% identity to that of SEQ ID NO: 22.

The antisense strand of any RNA duplex comprises a sequence that is complementary to the sense strand. As used herein, a “sequence that is complementary to the sense strand” will be able to form a duplex despite having a less than 100% complementarity to the sense strand if at least a portion of the sequence is able to form a duplex with the sense strand. In some embodiments, the antisense strand is at least 95% complementary to the sense strand. In other embodiments, the antisense strand is at least 97% complementary to the sense strand. In further embodiments, the antisense strand is at least 99% complementary to the sense strand. In yet further embodiments, the antisense strand is about 100% complementary to the sense strand. It will be appreciated that the degree of identity between the sense and antisense strands of the RNA duplex may be different than the degree of identity between the sense strand and the respective target sequence.

The two strands forming the RNA duplex may be different portions of one larger RNA molecule, or they may be separate RNA molecules. Where the strands of the RNA duplex are formed from separate RNA molecules, the RNA duplex may be a “small interfering RNA” (“siRNA”). Where the two strands are part of one larger molecule, and therefore are connected by a chain of nucleotides between the 3′-end of one strand and the 5′ end of the other strand of the RNA duplex, the RNA duplex is a “short hairpin RNA” (“shRNA”).

In some embodiments, the RNA duplex is a siRNA comprising a sense strand and an antisense strand. In one embodiment, the RNA duplex is a siRNA having a sense strand having the sequence of SEQ ID NO: 6, and an antisense strand having the sequence of SEQ ID NO: 7 (referred to herein as “si143” or “siRNA143”). In another embodiment, the RNA duplex is a siRNA having a sense strand having the sequence of SEQ ID NO: 14, and an antisense strand having the sequence of SEQ ID NO: 15 (referred to herein as “si136” or “siRNA136”). In a further embodiment, the RNA duplex is a siRNA having a sense strand having the sequence of SEQ ID NO: 22, and an antisense strand having the sequence of SEQ ID NO: 23 (referred to herein as “si205” or “siRNA205”).

In other embodiments, the RNA duplex is a shRNA comprising a sense strand and an antisense strand. In one embodiment, the RNA duplex is a shRNA comprising a sense strand having the sequence of SEQ ID NO: 6 (or a sense strand having at least 95% identify to that of SED IQ NO: 6), and an antisense strand having the sequence of SEQ ID NO: 7. In yet another embodiment, the shRNA has the sequence of SEQ ID NO: 8 (referred to herein as “sh143” or “shRNA143”). In another embodiment, the RNA duplex is a shRNA comprising a sense strand having the sequence of SEQ ID NO: 14 (or a sense strand having at least 95% identify to that of SED IQ NO: 14), and an antisense strand having the sequence of SEQ ID NO: 15. In yet another embodiment, the shRNA has the sequence SEQ ID NO: 16 (referred to herein as “sh136” or “shRNA136”). In a further embodiment, the RNA duplex is a shRNA comprising a sense strand having the sequence of SEQ ID NO: 22 (or a sense strand having at least 95% identify to that of SED IQ NO: 22), and an antisense strand having the sequence of SEQ ID NO: 23. In yet another embodiment, the shRNA has the sequence of SEQ ID NO: 24 (referred to herein as “sh205” or “shRNA205”).

In other embodiments, the at least one silencing nucleic acid is a nucleic acid selected from the group consisting of antisense RNA, DNA or mixtures thereof; a DNAzyme; and a ribozyme; which target at least one of a sequence of SEQ ID NO: 1, a sequence of SEQ ID NO: 9, or a sequence of SEQ ID NO: 17. Methods for the preparation and use of antisense RNA and DNA molecules, ribozymes and DNAzymes are known in the art and are described in, for example, Jakobsen et al, 2007, Retrovirology 4: 29-41, the disclosure of which is hereby incorporated herein by reference in its entirety.

The siRNA and shRNA described herein may be obtained using a number of techniques known to those of ordinary in the art. For example, the siRNA and shRNA can be chemically synthesized or recombinantly produced using method known in the art. The siRNA or shRNA can be chemically synthesized using appropriately protected ribonucleotide phosphoramidites and a conventional RNA/DNA synthesizer. The siRNA can be synthesized as two separate complementary molecules, while the shRNA may be synthesized with both the sense and antisense strands as a single molecule.

The RNA strands of the RNA duplex may have the same or a different number of nucleotides. In this regard, the RNA duplex may comprise one or more nucleotide overhangs. One or both strands of the siRNA may also comprise a 3′ overhang. As used herein, a “3′ overhang” refers to at least one unpaired nucleotide extending from the 3′ end of the duplexed RNA. In embodiments in which both strands of the siRNA are 3′ overhangs, the length of the overhangs can be the same or different for each strand. In one form, the 3′ overhang is the same on both strands. In one form, the 3′ overhang is two nucleotides, such as TT, on each end of the siRNA.

The two separate strands of any siRNA disclosed herein may be covalently connected, typically between the 3′-end of one strand and the 5′ end of the other strand forming the duplex structure, by a “linker.” Methods for the chemical linking of two separate RNA strands are known in the art and may be achieved, for example by introducing covalent, ionic or hydrogen bonds; hydrophobic interactions, van der Waals or stacking interactions; by means of metal-ion coordination, or through use of purine analogues.

Any of the RNA duplexes disclosed herein may be modified. In this regard, the sense and/or antisense strands of the RNA duplex may be chemically modified with 2′-OMe nucleotides, 2′-O-allyl nucleotides, 2′-deoxy-2′-fluoro nucleotides, 2′-deoxy nucleotides, 2′-O-(methoxyethyl (MOE) nucleotides, locked nucleic acid (LNA) nucleotides, phosphorothioate (PS) linkages, and combinations thereof. For example, modified siRNA or shRNA may comprise 2′-O-Me-purine or pyrimidine nucleotides such as 2′-O-Me-uridine nucleotides, 2′-O-Me-guanosine nucleotides, 2′-O-Me-cytosine nucleotides, 2′-O-Me-adenosine nucleotides, LNA nucleotides and mixtures thereof.

In other embodiments, the silencing nucleic acid may be a DNA polynucleotide which encodes the sense and/or antisense sequence of the siRNA or shRNA. Such DNA sequences may be inserted into vectors, such as plasmids, viral vectors, etc., to achieve expression of the siRNA or shRNA in the cell. In the case of siRNA, the sense and antisense strands of the siRNA may be expressed from the same or different vectors.

In another aspect, is a method of treating HIV infection in a subject. As used herein, “treating” means affecting a subject, tissue or cell to obtain a desired pharmacological and/or physiological effect and includes inhibiting the condition, i.e. arresting its development; or relieving, mitigating or ameliorating the effects of the condition i.e. cause reversal or regression of the effects of the condition.

As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject. In one embodiment, treating achieves the result of preventing or reducing HIV replication. Treating may also achieve the result of preventing reactivation of latent HIV1-virus in the recipient subject.

In one embodiment, treating HIV infection in a subject prevents or reduces HIV infection in a subject suffering from HIV infection. Thus, in one form, there is provided a method of preventing or reducing HIV infection in a subject. In one embodiment, preventing or reducing HIV infection in a subject comprises preventing or reducing HIV infection in a subject suffering from HIV infection. As used herein, the expression “preventing or reducing HIV infection in a subject suffering from HIV infection” refers to eliminating, reducing or delaying the production of infectious HIV virus in a subject already infected with HIV such that infection of uninfected tissue with HIV in the subject is prevented, reduced or delayed. In another embodiment, preventing or reducing HIV infection in a subject comprises preventing or reducing a productive HIV infection in a subject not suffering from HIV infection. As used herein, “preventing or reducing a productive HIV infection in a subject not suffering from HIV infection” refers to preventing or reducing development of an HIV infection of a subject who has not been previously infected with HIV.

As used herein, the term “subject” refers to a mammal that is susceptible to HIV infection, such as a human, mouse or primate. Typically, the mammal is a human (homo sapiens).

As used herein, the term “administering” means providing a composition, formulation, or specific agent to a subject in need of treatment.

The subject is administered an effective amount of the silencing nucleic acid. As used herein, an “effective amount” is an amount sufficient to cause inhibition of HIV gene expression. An affective amount can be readily determined by those skilled in the art based on factors such as body size, body weight, age, health, sex of the subject, ethnicity, and viral titers.

Any of the silencing nucleic acids disclosed herein (or combinations thereof) may be formulated as a pharmaceutical composition. The phrases “pharmaceutically acceptable” or “pharmacologically acceptable” refer to molecular entities and compositions that do not produce adverse, allergic, or other untoward reactions when administered to an animal or a human. For example, the silencing nucleic acid may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

The pharmaceutical compositions may comprise any of the silencing nucleic acids disclosed herein (or combinations thereof) in any concentration that allows the silencing nucleic acid administered to achieve a concentration in the range of from about 0.1 mg/kg to about 1 mg/kg. The pharmaceutical compositions may comprise the silencing nucleic acid in an amount of from about 0.1% to about 99.9% by weight. Pharmaceutically acceptable carriers include water, buffered water, saline solutions such as, for example, normal saline or balanced saline solutions such as Hank's or Earle's balanced solutions), glycine, hyaluronic acid etc.

The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol, etc.), carboxymethylcellulose and mixtures thereof, vegetable oils (such as olive oil), injectable organic esters (e.g. ethyl oleate).

The pharmaceutical compositions may comprise any of the silencing nucleic acids disclosed herein (or combinations thereof) in an encapsulated form. For example, the silencing nucleic acid(s) may be encapsulated by biodegradable polymers such as polylactide-polyglycolide, poly(orthoesters) and poly(anhydrides), or may be encapsulated in liposomes or microemulsion. Liposomes may be, for example, lipofectin or lipofectamine. Another example may comprise the silencing nucleic acid in or on anucleated bacterial minicells (Giacalone et al, Cell Microbiology 2006, 8(10): 1624-33). The silencing nucleic acid may be combined with nanoparticles.

The pharmaceutical composition may be formulated for oral administration. Solid dosage forms for oral administration may include, for example, tablets, dragees, capsules, pills, and granules. In such solid dosage forms, the composition may comprise at least one pharmaceutically acceptable carrier such as sodium citrate and/or dicalcium phosphate and/or fillers or extenders such as starches, lactose, sucrose, glucose, mannitol, and silicic acid; binders such as carboxylmethylcellulose, alginates, gelatin, polyvinylpyrrolidone, sucrose and acacia; humectants such as glycerol; disintegrating agents such as agar-agar, calcium carbonate, potato or tapioca starch, alginic acid, silicates, and sodium carbonate; wetting agents such as acetyl alcohol, glycerol monostearate; absorbants such as kaolin and bentonite clay; and/or lubricants such as talc, calcium stearate, magnesium stearate, solid polyethylene glycol, sodium lauryl sulfate, and mixtures thereof. Liquid dosage forms for oral administration may include, for example, pharmaceutically acceptable emulsions, solutions, suspensions, syrups and elixirs. Liquid dosages may include inert diluents such as water or other solvents, solubilizing agents and/or emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as, for example, cottonseed oil, corn oil, germ oil, castor oil, olive oil, sesame oil), glycerol, tetrahydrofurfuryl alcohol, polyethylene glycols and fatty acid esters of sorbitan, and mixtures thereof.

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salycilate, 5-methoxysalicylate, homovanilate). The silencing nucleic acid may be delivered combined with minicells or nanoparticles.

Any of the silencing nucleic acids disclosed herein (or combinations thereof) may be administered to a subject by administering cells which comprise the silencing nucleic acid (i.e. the cells have been pre-treated with one or more silencing nucleic acids). Cells which comprise the silencing nucleic acid may be cells into which the silencing nucleic acid has been directly introduced, or may be cells into which a nucleic acid which encodes, for example, the sense and antisense strand of the siRNA or shRNA molecule, has been introduced. Thus, in one aspect, there is provided a cell that comprises a silencing nucleic acid which targets a sequence in the region from about position 143 to about position 161 of the 5′ LTR of HIV-1. Thus, in one aspect, there is provided a cell that comprises a silencing nucleic acid which targets a sequence in the region from about position 136 to about position 154 of the 5′ LTR of HIV-1. Thus, in one aspect, there is provided a cell that comprises a silencing nucleic acid which targets a sequence in the region from about position 205 to about position 233 of the 5′ LTR of HIV-1. The cell which comprises the silencing nucleic acid may be a stem cell, such as an embryonic stem cell, an adult stem cell, or an induced pluripotent stem cell (IPSC). Adult stem cells may be hematopoietic stem cells (HSC), mesenchymal stem cells, neural stem cells. Typically, the adult stem cell is a hematopoietic stem cell. The cell may be a differentiated or partially differentiated cell, such as a CD4+ T cell (or mature forms of CD4+ T cells such as, for example, CD4+ memory cells), a myeloid cell (or more differentiated myeloid cells such as macrophages and dendritic cells), or precursors thereof such as a thymocyte. The cells may be obtained from the subject (i.e. ex vivo) or from an alternative source. Methods for the preparation of stem cells, differentiated cells and precursors thereof, are well known in the art. Methods suitable for the transfer of nucleic acids into mammalian cells in vitro are known in the art and include the use of liposomes, electroporation, microinjection, cell fusion, DEAE-dextran, the calcium phosphate precipitation method, nanoparticles or minicells.

Any of the silencing nucleic acids disclosed herein (or combinations thereof) acid may be introduced into cells by conjugating a moiety to enhance its cellular absorption, as well as in some cases targeting it to a particular tissue or facilitating uptake by specific types of cells. For example, a hydrophobic ligand conjugated to the dsRNA may facilitate direct permeation of the cellular membrane, or a ligand or moiety may be attached which facilitates targeting or receptor-mediated endocytosis or cellular uptake. Examples of such ligand or moieties include: cholesterol; other lipophilic compounds such as: 1-pyrene butyric acid, 1,3-bis-O-(hexadecyl)glycerol, and menthol; folic acid; polyethylene glycols; carbohydrate clusters; cross-linking agents; porphyrin conjugates; delivery peptides; monoclonal antibodies against a target molecule; aptamers capable of binding to a target molecule.

Any of the silencing nucleic acids disclosed herein (or combinations thereof) may be introduced into cells using recombinant viral vectors. The recombinant viral vectors typically comprise sequences encoding the silencing nucleic acid and any suitable promoter for expressing the silencing nucleic acid. Suitable promoters are known in the art and their selection is well within the skill in the art. When the silencing nucleic acid is an RNA duplex, the RNA duplex can be expressed from a recombinant viral vector either as two separate, complementary RNA molecules, or as a single RNA molecule with two complementary regions. Any viral vector capable of accepting the coding sequences for the RNA duplex molecule(s) to be expressed can be used, for example vectors derived from adenovirus (AV); adeno-associated virus (AAV); retroviruses (e.g. lentiviruses (LV), Rhabdoviruses, murine leukemia virus); herpes virus, and the like. Selection of recombinant viral vectors suitable for use in the invention, methods for inserting nucleic acid sequences for expressing the duplex RNA into the vector, and methods of delivering the viral vector to the cells of interest are within the skill in the art.

Also provided is an article of manufacture and a kit, comprising at least one of (i) a silencing nucleic acid which targets a sequence from about position 143 to about position 161 of the 5′ LTR of HIV-1, (ii) a silencing nucleic acid which targets a sequence from about position 136 to about position 154 of the 5′ LTR of HIV-1, and/or (iii) a silencing nucleic acid which targets a sequence from about position 205 to about position 223 of the 5′ LTR of HIV-1. The kit may comprise a container, where the container may be a bottle comprising the antisense nucleic acid in oral or parenteral dosage form, each dosage form comprising a unit dose of the silencing nucleic acid. For example, siRNA in an amount for example from about 1 nanomolar (nM) to about 100 nM, from about 2 nM to about 50 nM, from 2 to about 10 nM. The kit will further comprise printed instructions. The article of manufacture will comprise a label or the like, indicating treatment of a subject according to the present method. In one form, the article of manufacture may be a container comprising the silencing nucleic acid in a form for oral or parental dosage. For example, the siRNA may be in the form of an injectable solution in a disposable container.

In some embodiments, the kit comprises at least one of si143, si136, or si205. In other embodiments, the kit comprises at least two of si143, si136, or si205. In yet other embodiments, the kit comprises all three of si143, si136, or si205. In some embodiments, the kit comprises at least one of sh143, sh136, or sh205. In other embodiments, the kit comprises at least two of sh143, sh136, or sh205. In yet other embodiments, the kit comprises all three of sh143, sh136, or sh205. In some embodiments, the kit comprises any combination of siRNAs or shRNAs.

It will be understood that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In order to exemplify the nature of the present invention such that it may be more clearly understood, the following non-limiting examples are provided.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

In order to evaluate the potential of promoter-targeted sequences to induce viral suppression, a panel of siRNAs targeting the HIV 5′LTR promoter region were designed and tested for induction of TGS. The classic characteristic of TGS is formation of heterochromatin or closed chromatin, including deactylation and methylation of specific residues in histone 3 molecules.

Materials and Methods

RNA Duplexes

Double-stranded RNA duplexes were designed to target the HIV 5′LTR region. All RNA duplexes were synthesized by Invitrogen. Each siRNA construct was 19 bp in length and was designed with a 3′TT overhang. Also included was an siRNA targeting the Tat exon, previously described PromA, PromB, PromC and PromD sequences and a control siRNA targeting SIV (Suzuki et al, 2008, J Biol Chem 283: 23353-23363; Suzuki et al, 2005, J RNAi Gene Silencing 1: 66-78).

Cell Culture

Media and reagents for cell culture were purchased from Gibco. 293T, MAGIC-5, HeLa T4+ and the HeLa CMV-3LTR1-4 cells were grown in Dulbecco's modified Eagle's medium (DMEM) containing 10% fetal calf serum, 5 U/ml penicillin and 50 mg/mL streptomycin (supplemented DMEM) and incubated at 37° C. in a humidified incubator with 5% CO2. HeLa T4+ and HeLa CMV-3LTR1-4 cells were maintained under selection with G418 (500 μg/mL) and Hygromycin (300 μg/mL), respectively.

HIV-1 GP-Pseudotyped Lentivirus Generation, Infection and siRNA Transfection

Plasmids used to generate recombinant lentiviral vectors included an HIV-1 pNL4.3Δenv provirus plasmid containing a GFP reporter gene fused to the 3′end of the MA of the gag region, the VSV-G envelope expression plasmid and a helper PAX-2 plasmid containing the HIV-1 gag and pol regions (Aggarwal et al, 2012, PLoS pathogens 8: e1002762). All plasmids were a kind gift from Dr. Stuart Turville (Kirby Institute, University of New South Wales). HIV-1 GP-pseudotyped lentivirus was produced as described previously (Kong et al, 2006, Proc Natl Acad Sci USA 103: 15987-15991). Briefly, 293T cells were co-transfected with the abovementioned plasmids using the calcium phosphate method, incubated overnight, then washed the following day and fresh medium was added. Supernatants were harvested 48 h later, filtered through a 0.45-μm syringe filter, aliquoted, and stored at −80° C. Initial titering and analysis of HIV viral stocks utilized the TZM-bl indicator cell line as previously described (Turville et al, 2008, PloS one 3: e3162). RT activity of newly produced HIV-1 GP pseudotyped lentivirus was determined to be 2250 pg/μL using the previously described method (Suzuki et al, 1993, J Virol Methods 44: 189-198).

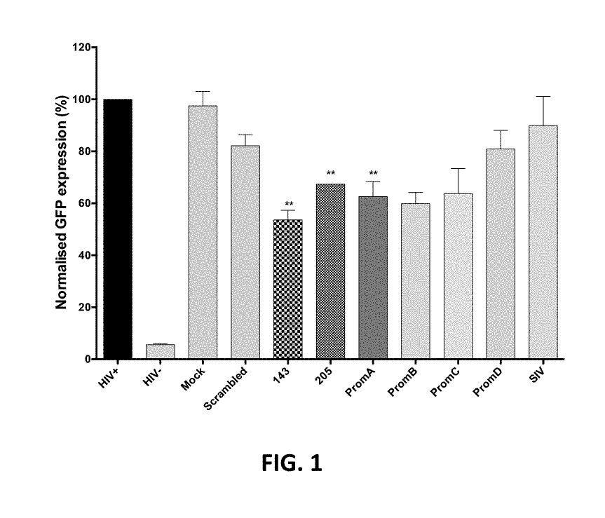

HIV-1 GP-pseudotyped lentivirus with a TCID50 of 5×10⁴ (50 μL) was added to 1×10⁵ pre-seeded 293T cells for 6 h, washed once and fresh culture medium was added. The cultures were then transfected with 100 pmol of each siRNA from the panel of siRNAs shown in FIG. 1, using Lipofectamine2000 as per the manufacturer's instructions (Invitrogen, Life Technologies). Transfected cultures were incubated for 48 h and then analyzed for GFP expression using flow cytometry.

Live HIV Infection and siRNA Transfection

Cell cultures for time course experiments using replication competent virus were seeded with 5×10⁴ MAGIC-5 cells (HeLa cells stably transfected with CD4, CCR5 and CXCR4) or HeLa-T4+ cells, incubated overnight and then transfected with 50 pM of the appropriate siRNA panel. The following day, MAGIC-5 siRNA-transfected cultures were infected with HIV-1 strain BaL, or HeLa-T4+ siRNA-transfected cultures were infected with HIV-1 strain SF162, using 100 pg/μL and 140 pg/μL, respectively. Supernatants were harvested over a time course up to 15 days for analysis of virus production using the reverse transcriptase (RT) assay described below. Cultures for the trichostatin A (TSA) drug reactivation experiments were seeded with 1×10⁵ HeLa-T4+ cells, incubated overnight and then transfected with 80 pM of siRNAs 143, 143T, PromA, Scram, M2 or mock-transfected. The following day HeLa-T4+ siRNA-transfected cultures were infected with HIV-1 strain SF162 using 140 pg/μL and the infection was allowed to proceed for 6 days prior to drug treatment. Cultures for the ChIP experiment were seeded with 2×10⁵ HeLa-T4+ cells, incubated overnight and then infected with HIV-1 strain SF162 using 1000 pg/μL and the infection was allowed to proceed for 3 days. The infected cultures were then transfected with 300 pM siRNAs 143, 143T, PromA, 136T, 205S or mock-transfected for 48 h prior to harvest for the ChIP assay. Transfection of 293T cells, MAGIC-5 cells and HeLa T4+ cells used for the 3′PTGS studies were performed using Lipofectamine2000 (Invitrogen, Life Technologies) according to the manufacturer's instructions. All other transfections were performed using RNAiMax (Invitrogen, Life Technologies) according to the manufacturer's instructions.

Viral Quantitation

Reverse transcriptase (RT) activity in culture supernatants was determined as described previously (Suzuki et al, 1993).

HIV-1 mRNA was quantified using a real-time RT-PCR assay specific for HIV-gag as previously described (Suzuki et al, 2005 supra). Briefly, RT-PCR reactions were performed with SuperScript One-step RT-PCR (Invitrogen) using 0.4 μM of both sense and anti-sense primers, and 0.1 μM of sequence-specific fluorogenic Taqman probe. Standard curves were constructed using genomic HIV plasmid pNL4-3 for HIV-1 and a TA-cloned PCR fragment of beta-actin (Invitrogen, Mount Waverley, Australia). The primers and probes used are provided by SEQ ID NO: 25, SEQ ID NO: 26, SEQ ID NO: 27, SEQ ID NO: 28, SEQ ID NO: 29, and SEQ ID NO: 30.

Assessment of PTGS Induced by U3 Region-Targeted siRNAs

A HeLa T4+ cell line stably expressing high levels of the HIV-1 3′-LTR region overlapping into the coding region of nef, designated CMV-3LTR1-4 (Suzuki et al, 2008, supra), was utilized to assess the extent of PTGS contribution to silencing induced by promoter-targeted siRNAs. Briefly, HeLa T4+ CMV-3LTR1-4 cells were transfected with the panel of promoter-targeted siRNAs (100 pM) and variants at 48 h following transfection cells were harvested and expression of the 3′LTR was measured by SYBR-Green based quantitative Real-Time PCR assays, as previously described (Suzuki et al, 2008, supra using the primer set of NUAf, (SEQ ID NO: 31), and Chips2r (SEQ ID NO: 32). Two positive controls were included, siRNA Nef366, which targets the U3 region upstream of siRNA 143 and siRNA PromA (Yamamoto et al, 2006, Blood 108: 3305-3312), and siRNA PolyA, which targets a sequence downstream of the R region transcription start site and both of which induce the PTGS pathway to result in mRNA degradation. Data were normalized to a GAPDH control and statistical comparisons were made between the mock-transfected cultures and siRNA-transfected cultures.

Drug Treatments

HeLa-T4+ cell culture experiments investigating virus reactivation using the HDAC inhibitors trichostatin A (TSA; 50 nM) and SAHA (2.5 μM), were treated for 24 h on day 8 during the time course HIV-1 infection and siRNA-transfection as described above. Positive controls included siRNA-transfected and mock-transfected cultures treated with TNFα (10 ng/mL) for 24 h. DMSO was added to the untreated cultures in the same concentration it is present in the final concentrations of the drugs. All cultures were harvested at the same time and analyzed for intracellular viral mRNA levels by RT-PCR as described above.

Chromatin Immunoprecipitation (ChIP) Assays

HeLa T4+ cells (5×10⁵) were seeded into T-25 flasks and 24 h later infected with HIV-1 strain SF162 as described above. At day 3 post-infection, the cultures were transfected with 300 pmol of each of siRNAs 143, 143T, the current lead siRNA PromA, 136T, 205S or mock-transfected as a control culture as described above. Following 48 h post-transfection, cultures were harvested for the ChIP assay using the EZ Magna A/G ChIP Kit (Millipore, Australia) following manufacturer's instructions. Pellets were sonicated for 20 minutes (1 min off, 1 min on) in a COVARIS 5 sonicator at 5% Duty cycle: intensity 4, Burst/cycles 200, and protein isolated according to manufacturer's instructions. Immunoprecipitations were performed using 5 μg/ml of each antibody for each 2×10⁶ cell equivalents.

Statistical Analysis

Pseudotyped virus data are shown as mean and SD and RT values are given as mean and SEM and both data sets were tested for significance using a paired, two-tailed t-test. NEF 3′LTR, Drug activation and ChIP data were tested for significance using a non-parametric Mann-Whitney test and are given as mean±SEM. A p-value of <0.05 was considered statistically significant. All analyses were performed using Graphpad Prism Version 6.0 (Graphpad Software, San Diego, Calif.).

Results

Candidate siRNA can Induce HIV Suppression in a Pseudotyped Virus System

In order to rapidly screen an siRNA panel for potential HIV suppression, a VSV-G pseudotyped GFP expressing HIV-1 infected 293T cell model was used, which allowed for experiments to be performed in a lower bio-containment setting than that required for live HIV-1. Recombinant lentiviral VSV-G pseudotyped HIV-1 containing a GFP reporter were generated and changes in GFP expression were measured by flow cytometry following transfection of each member of the siRNA panel as a read out to determine their potential for HIV suppression.

Flow cytometry analysis confirmed that the siRNAs PromA, PromB, PromC and PromD were functional in reproducing the expected HIV suppressive effect in the pseudotyped HIV reporter system, with siPromA- and siPromB-transfected cells showing significantly lower GFP expression than siPromC- and siPromD-transfected cells when compared to HIV-1 control cells (both p=≦0.008) (FIG. 1). Screening of the siRNA panel revealed that siRNA 143 and 205 showed significant reduction in GFP expression and suppression of pseudotyped HIV-1 compared to controls (both p=≦0.008). Importantly, siRNA targeting SIV and scrambled siRNA controls did not show any significant decrease in GFP expression (FIG. 1). These data indicate that siRNAs 143 and 205 targeting the U3 region have the potential to suppress virus transcription in a pseudotyped single round HIV-1 infection model. siRNA 143 has a sense strand with the sequence of SEQ ID NO: 2 and an antisense strand with the sequence SEQ ID NO: 3.

siRNA 143, 136T and 205S Targeting the U3 Region can Induce HIV Suppression Using Live Virus Strains

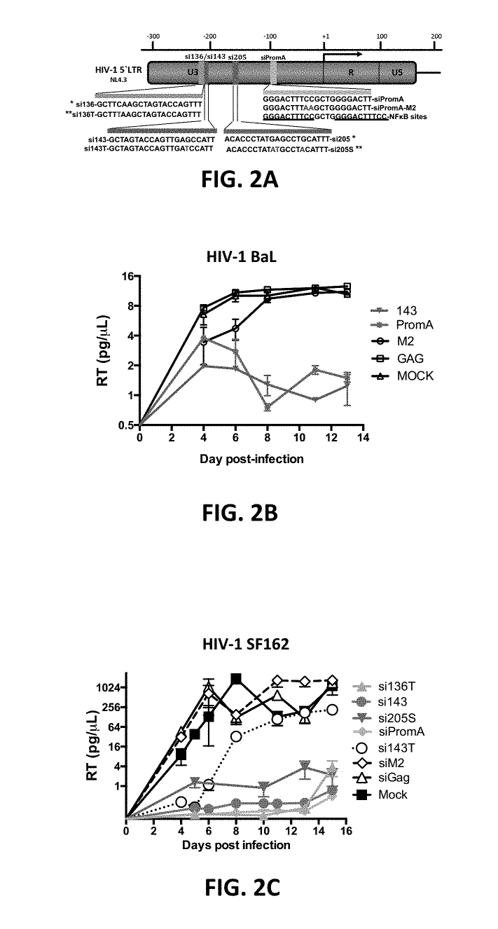

In order to confirm the observed pseudotyped HIV suppression using a replication competent HIV-1 strain, MAGIC-5 cells were infected with HIV-1 subtype B strain BaL and reverse transcriptase (RT) activity was measured over a prolonged time course of infection. siRNA 143 potently suppressed live HIV-1 strain BaL, to a level comparable with PromA (FIG. 2B), which was a about 12 fold reduction in RT activity compared to the infected mock-transfected cells. The siRNA 143 is located in the U3 region upstream of PromA (FIG. 2A).

The suppressive effect of siRNA 143, 136T and 205S was confirmed using HeLa T4+ cells infected with HIV-1 subtype B strain SF162 and again measuring RT activity over a prolonged time course of infection (FIG. 2C). The siRNAs 136T and 205S are located in the U3 region upstream of PromA (FIG. 2A) and are nearly identical to the HIV-1 Bal strain 136 and 205, with the exception of a single T nucleotide change in 136, designated 136T, and three A nucleotide changes in 205, designated 205S, in the HIV-1 SF162 strain. The siRNA 143 target is identical to the SF162 strain. To determine whether a single mismatch is critical in reducing the suppressive effect, we also included siRNA 143T, which has a single T mismatch compared to both BaL and SF162 strains. This mismatched siRNA 143T corresponds to the SF162 3′LTR sequence available in Embank (accession number M65024.1). Also included were the sequence specificity controls siPromA-M2 and siPromA-Scram. Both of the completely matched siRNAs, 143, 136T, 205S and PromA, potently suppressed productive infection with HIV-1 strain SF162, with greater than 1000-fold reduction in RT activity compared to mock-transfected cells. Interestingly, 143T suppressed virus infection to a level comparable to 143 and PromA up to day 6 post-infection, but was then unable to maintain virus suppression. siRNAs with SF162 strain mismatches showed no significant reduction in RT activity compared to infected mock-transfected cells (data not shown).

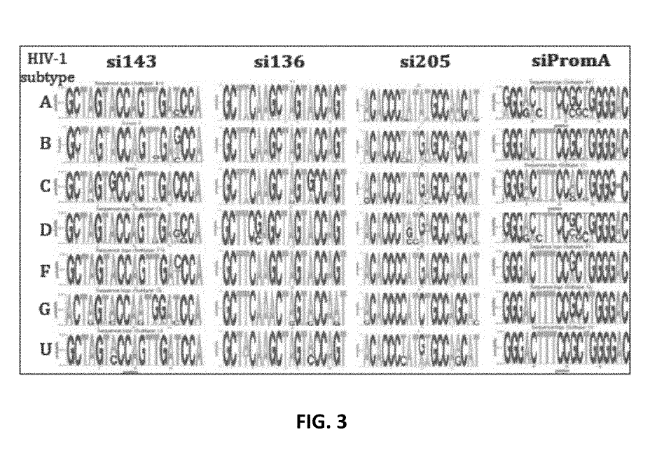

To determine sequence conservation of siRNAs 143, 136 and 205, we performed a sequence alignment across HIV-1 subtypes A through G and U and generated sequence logos (FIG. 3) (Crooks, Hon, Chandonia, & Brenner, 2004, Genome Res 14: 1188-90; Schneider, & Stephens, 1990, Nucleic Acids Res 18, 6097-100). SiRNA 143 sequence conservation showed 94.9% median identity over 19 nucleotide positions with an identity range of 9/19 nucleotides having >95% conservation, 13/19 nucleotides having >90% conservation and 18/19 nucleotides having >80% conservation. As previously reported siPromA is highly conserved across all subtypes, with the exception of a one bp deletion in subtype C, and showed 98.4% median identity over 19 nucleotide positions (FIG. 3).

Limited Contribution of PTGS Activity was Observed with Potent Suppressive siRNA Candidates

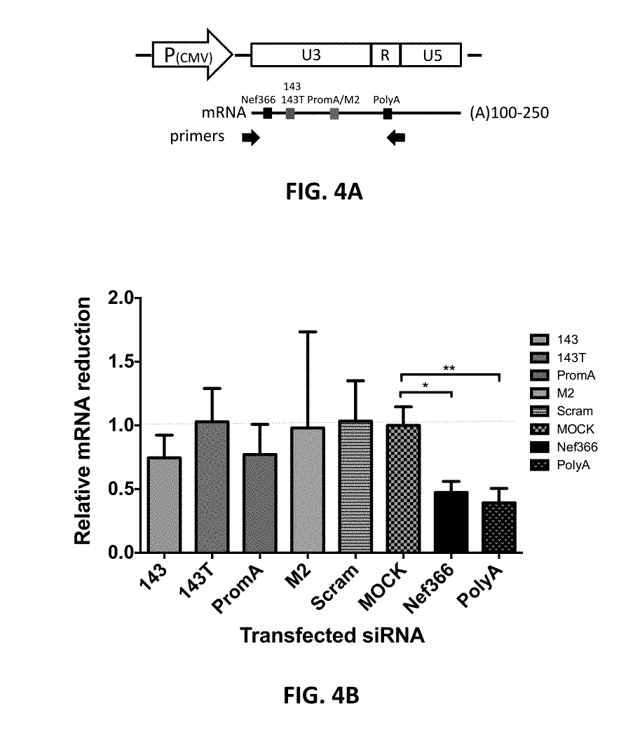

HIV-1 proviral DNA contains two LTR regions, the 5′LTR and 3′LTR, which are identical in sequence. Following integration the 5′LTR functions as the promoter for transcription of the HIV-1 genome, while the 3′LTR provides nascent viral RNA polyadenylation and encodes the Nef accessory protein (Klaver & Berkhout, 1994, Journal of Virology 68: 3830-3840). SiRNA sequences targeting the 5′LTR region could therefore result in post transcriptional gene silencing via the sequences in the 3′LTR region, contributing to the suppressive effect observed. To investigate this possibility, a HeLa T4+ cell line stably expressing the 3′LTR sequence under the control of the CMV promoter, designated CMV3′LTR1-4 (which includes part of the nef sequence), was transfected with siRNAs 143, 143T, and PromA. We also included the sequence specificity controls siPromA-M2 and siPromA-Sc, as well as two positive control siRNAs, Nef366 targeting the U3 region (Yamamoto et al, 2006, Blood 108: 3305-3312) and PolyA, which targets a sequence downstream of the transcription start site and are both potent inducers of PTGS, thereby resulting in mRNA degradation. The results are shown in FIG. 4.

As can be seen from FIG. 4, following RT-PCR analysis, no significant reductions in the 3′LTR mRNA levels expressed by any candidate siRNA-transfected cultures (143, 143T, and PromA) or the sequence specificity controls (M2 and Sc) compared to the mock-transfected cultures (FIG. 4) was observed. We did observe a significant mRNA reduction in the cultures transfected with the two positive control siRNAs, PolyA (p=0.009) and Nef366 (p=0.028) (FIG. 4). These data demonstrate that the PTGS pathway has a limited contribution to the potent HIV-1 suppressive U3 targeted siRNA 143, 143T, and PromA.

Reactivation of HIV-1 Transcription by Treatment with HDAC Inhibitors was Observed in HIV-1 Cultures Suppressed by siRNA Candidates

To further explore the mechanism responsible for the induction of HIV-1 suppression following transfection with siRNA 143, the association between histone deacetylation and reduction of virus replication using the histone deacetylase (HDAC) inhibitor trichostatin A (TSA), which selectively inhibits type I and type II HDACs was investigated. These events mimic the characteristics of one type of latent HIV-1 infection, which is associated with recruitment of HDACs to the 5′LTR region resulting in H3 deacetylation, a classic heterochromatin marker. This is a precursor to methylation of certain residues. It is known that TGS in general, and that induced by PromA in particular, is associated with induction of heterochromatin and deacetylation of histones. Further, it is also described that the silencing of virus either by siRNA induced TGS or other forms of latency, such as HIV-1, can be reversed by treatment with HDAC inhibitors. Cultures that were transfected with siRNA 143 were therefore treated with TSA, or another type I and II HDAC inhibitor suberoylanilide hydroxamic acid (SAHA, also known as Vorinostat) that is currently been tested in clinical trials aiming to purge the latent HIV (http://aidsinfo.nih.gov/clinical-trials NCT01319383 and NCT01365065), or with TNFα, a potent reactivator of latent HIV-1, which acts via the RelA/NF-κB pathway.

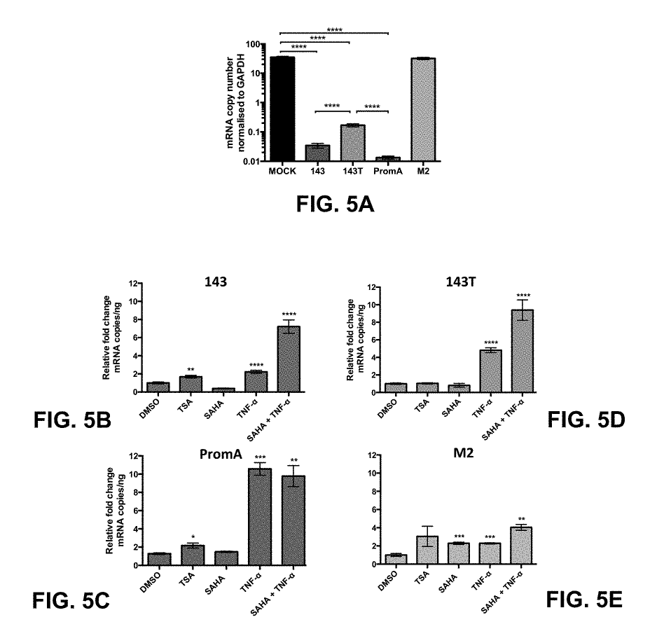

Following HIV-1 infection for 6 days and then transfection of siRNAs 143, 143T, PromA, M2 or Scram, cultures were treated with TSA, SAHA or TNFα, or a combination of SAHA and TNFα for 24 h, then intracellular viral mRNA levels were analyzed by RT-PCR. The results are shown in FIG. 5. Potent HIV-1 suppression is shown in cells transfected with 143, 143T and PromA, compared to mock and M2 controls (FIG. 5A). As can be seen from FIGS. 5B through 5E, only the HIV-1 infected cultures suppressed by transfection with siRNAs 143 and PromA showed two-fold increases in mRNA expression following TSA activation (p≦0.002 and p≦0.03, respectively) and were therefore reactivated from a latent state (FIG. 5). Neither the single mismatched siRNA 143T, nor the specificity control siRNA M2, showed any significant increase in viral RNA expression following TSA treatment compared to untreated transfected cultures. Interestingly, SAHA treatment did not increase the mRNA expression in the HIV-1 suppressed siRNA 143 and PromA transfected cultures, but did significantly increase mRNA levels two-fold in M2-transfected cultures compared to untreated transfected cultures (both p≦0.0002) (FIG. 5). The potent activator, TNFα, resulted in an significant increase of mRNA levels across the entire panel of siRNAs, most markedly in siRNA PromA treated cultures (11-fold, p≦0.002), then 143T (5-fold, p<0.0001), M2 (about 2 fold, p≦0.002) and 143 (about 2-fold, p<0.0001). The combination treatment of SAHA and TNFα resulted in a synergized activation of HIV-1 mRNA levels, again with significant increases observed in all siRNA transfected cultures (PromA 10-fold, p≦0.002); 143T 9-fold, p<0.0001; 143 7-fold, p<0.0001 and M2 4-fold, p≦0.002)(FIG. 5). Cultures transfected with siRNA M2 showed an increase across most of the treatments, which is potentially the result of both non-specific and specific activation of transcription, considering that this siRNA does not suppress the virus. Together, these data suggest that silencing induced by siRNA 143 involves histone deacetylation through the activity of HDACs type I and/or type II, but that the epigenetic changes are relatively resistant to reversal by some HDAC inhibitors.

Markers of Heterochromatin were Observed in HIV Cultures Suppressed by siRNA Candidate 143

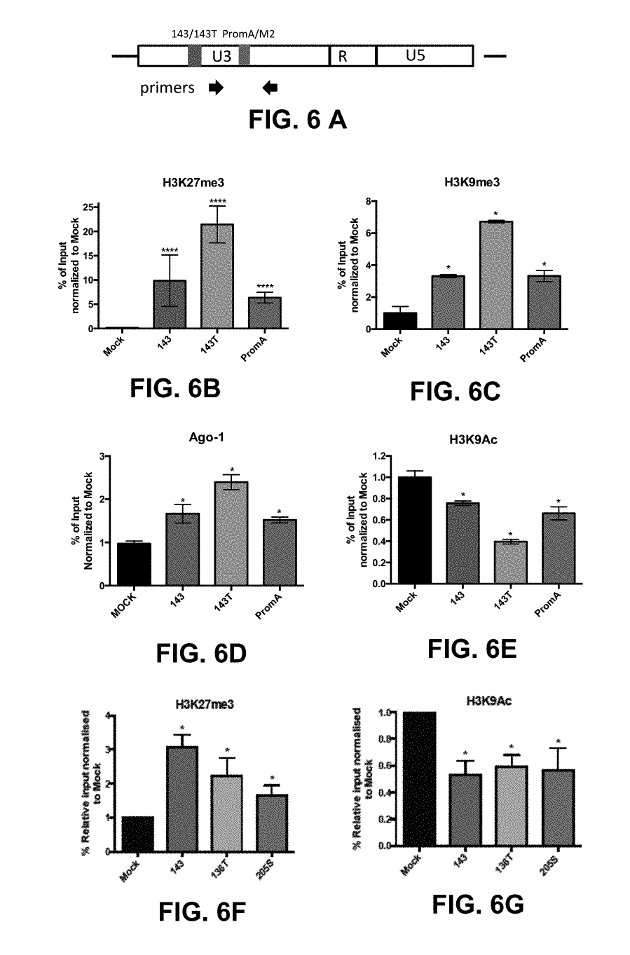

In order to determine whether the HIV suppression observed following transfection by siRNA 143 was associated with characteristic heterochromatin markers, we performed ChIP assays. Following HIV-1 infection for 3 days and siRNA transfection with 143, 143T, 136T, 205S, PromA or mock-transfection for 48 h, we analyzed alterations in histone methylation (H3K27me3 and H3K9me3), Argonaute 1 (Ago1) protein recruitment and histone acetylation status (H3K9Ac). The results are shown in FIG. 6.

As can be seen from FIG. 6, significant alterations were observed in the histone methylation marker, H3K27me3, in siRNA 143, 143T and PromA-transfected cultures that were increased greater than about 5 fold for 143- and PromA-transfected cultures (p<0.0001) and more than about 20 fold in 143T (all p<0.0001) compared with the mock-transfected cultures (FIG. 6B). Although the siRNA 143T has a one bp mismatch compared to the sequenced SF162 target strain, we did observe virus suppression up to day 6 post-infection, which then reverted to active virus replication (FIG. 2C). This early, but non-sustained, viral suppression is one explanation for the observed increase in H3K27me3 143T-transfected cultures at the day 3 post-infection time point. Both 136T and 205S-transfected cultures also showed H3K27me3 enrichment with >2-fold increases compared to mock-transfected cultures (both p<0.03) (FIG. 6F). Similarly, the H3K9me3 heterochromatin mark was reported to increase >3-fold in 143 and PromA-transfected cultures and >6-fold in the 143T-transfected cells (FIG. 6C). Ago1 recruitment was observed in all siRNA-transfected cultures (143, 143T and PromA) compared to mock-transfected cells (p<0.03) (FIG. 6D). Histone acetylation status (H3K9Ac) was reported to decrease in all siRNA-transfected cultures (143, 143T, 136T, 205S and PromA) compared to mock-infected cells (FIGS. 6E, 6G).

Efficacy of sh143, sh136 and sh205 when used alone or in combination with each other in LV transduced CD4+ T cells and CD34 HSC, across a range of viral subtypes.

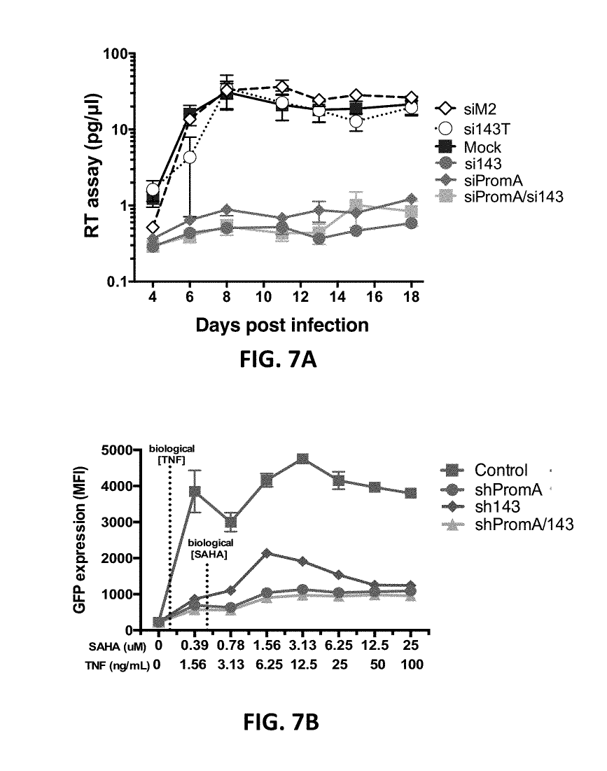

1a) LV vectors will be constructed by cloning sh143, sh136, and sh205 into a vector backbone. Effective expression and processing to siRNAs will be confirmed by PCR. Constructs comprising sh143, sh136, and/or sh205 alone or in combination with each other have already been synthesized, screened and shown to effectively suppress virus (FIG. 7B). Compositions, formulations, and/or constructs include sh143 alone; sh136 alone; sh205 alone; sh143 and sh136 combined; sh143 and sh205 combined; sh136 and sh205 combined; and sh143, sh136, and sh205 combined.

1b) Jurkat and Molt-4 CD4+ T cell lines will be infected with HIV-1 and several days later transduced with constructs comprising one or more of sh143, sh136, and/or sh205, and the dynamics of the viral infection will be monitored initially using reverse transcription (RT) assays in supernatants as shown in FIG. 7B and by cell associated viral RNA assays for gag and spliced tat. This will be repeated with a range of primary isolates from each major subtype of HIV-1 to confirm efficacy of the constructs across the inherent subtype variation of HIV-1.

1c) Primary CD4+ T cells will be obtained from healthy controls and infected with HIV-1 or from HIV-infected patients on cART.

1d) Latency model macrophage cells lines, U1 and U937, will be transduced with LVs described herein in the presence of Vpx to increase transduction efficiency and the experiments and analysis performed as in b). Results will be confirmed in monocyte derived macrophages and dendritic cells.

1e) All LV transduced cultures in b)-c) will be monitored for off target effects by monitoring cell surface phenotype, proliferation and viability as well as screening for off target effect such as the production of interferons (IFN) and interferon stimulated genes (ISGs) as previously described. CD34+ HSCs will be transduced and their ability to produce normal ratios of colony forming units in vitro will be monitored.

1f) Additionally, transduced CD4+ T cells from healthy controls and from HIV infected patients will be expanded using the Wave bioreactor technology by protocols already established in the art and used in the GMP synthesis of transduced CD4+ T cell preparations for reinfusion into patients in their phase 1 trials. The resulting expanded CD4+ T cell population will be monitored for their ability to suppress HIV, but also to produce cytokines and proliferate in response to T cell and co-receptor ligation (CD3/CD28).

1g) The lead candidates from LVs 1-v will be identified by an algorithm based on the results of the above sets of experiments to define those with the greatest ability to suppress the greatest number of different virus isolates for longest in the absence of off-target effects. The lead candidates will be then tested for their ability to inhibit viral reactivation against a range of inflammatory, immunological and homeostatic drivers in cell lines carrying latent virus such as J-Lat cells and primary CD4+ T cells infected and then silenced by LV transduction with shRNAs. Stimuli treatments will include; inflammatory cytokines, TNF, IL-1, IFN-α, Toll like receptor ligands, including LPS and CpG; immunological T cell stimulation with anti-CD3/anti-CD8, anti-CD3 in the presence of APC; and homeostatic cytokines, IL-2, IL-7 and IL-15.

1h) Efficacy of the lead LV expressing single or dual compositions and/or construct will then be assessed in vivo in the BLT humanized mouse model using CD34+ HSC transduced with the lead candidate or control empty constructs and challenged with HIV. The NOD-SCID, common gamma chain−/− (NSG)-humanized BLT mouse model is established and readily available at the UCLA AIDS Institute, USA by CIE, who will oversee all humanized mouse model experiments. CD34+ HSC will be prepared from foetal liver tissue and BLT mice will be generated as described. CD34+ HSCs will be transduced with the lead shRNA LV construct or empty vector using a standard RetroNectin-based method, which results in transduction efficiencies of about 60%. Briefly, myeloablated NSG mice will be implanted with foetal liver and thymus under the kidney capsule, and then injected with CD34+ HSCs, which engraft in the bone marrow. This model provides robust multi-lineage reconstitution of human hematopoietic cells, including functional T cells, macrophages, dendritic cells (DCs) and plasmacytoid DC (pDC). Cohorts of 25-30 BLT mice are routinely generated using the same donor tissue and each experimental condition typically has 5-8 mice per group, so mouse numbers will not limit the proposed studies. Twelve weeks after reconstitution, mice will be screened for human(h) cell engraftment and GFP expression to assess transduction of the construct by retro-orbital bleeding. The white blood cell fraction will be stained with antibodies against hCD45+, hCD3+(for T cells), hCD4+, hCD8+ and hCD14 (for myeloid cells), hCD19⁺ (B cells), and hCD56 (NK cells) for FACS analysis as described. If ≧20% of hCD4+ T cells demonstrate GFP expression, indicating carriage and expression of the construct, HIV-1 challenge experiments will proceed. BLT mice will be infected with R5-tropic HIV-1 strain NFN-SX by intra-peritoneal injection with 200 μg of HIV-1 p24 in a 100 μL volume. Blood will be collected at weeks 2-16 post infection for plasma viral load (pVL) analysis and hCD4+/hCD8+ ratios. At week 16 post-infection, mice will be sacrificed and the ratio of hCD4+/hCD8+, pVL, and HIV-1 p24 protein expression in CD4+ T cells in blood, bone marrow and peripheral lymphoid organs will be assessed by flow cytometry. We will measure cell associated HIV RNA in CD4+ T cells, CD14+ macrophages, and DCs, using cells obtained from the spleen.

We will assess the extent of viral reactivation within the latently infected CD4+ T cell population following stimulation with immobilized CD3 and CD28 antibodies for 3 days and then measure both p24-Gag expression and cell associated viral spliced-tat and unspliced gag and 5′LTR mRNA using quantitative RT-PCR. We expect to see an impact on the latently infected population using the lead LV construct, compared with the empty vector control. If we observe a reduction in reservoir size, we will proceed to assess heterochromatin formation in the HIV-1 promoter region by ChIP assays for methylation of H3K9, H3K27, histone deacetylation and recruitment of histone methyltransferases as previously performed.

The lead candidate will be the LV construct which fulfils the outcomes of b)-g). In vivo studies are expected to confirm control of virus and maintenance of CD4+ T cells counts, with viral reservoir in the periphery and tissues, and provide preliminary toxicity data.

It is believed that silencing nucleic acids which target a sequence from about position 136 to about position 154 of the 5′ LTR of HIV-1 or a sequence from about position 205 to about position 223 of the 5′ LTR of HIV-1 are as effective as those that target a sequence from about position 143 to about position 161 of the 5′LTR of HIV-1. Moreover, it is believed that treatment with a combination of silencing nucleic acids which target two or more of any of the above 5′LTR target sequences is at least as effective as a treatment using a single silencing nucleic acid that targets any one of the above-identified 5′LTR target sequences.

### SUMMARY

Without wishing to be bound by any particular theory, it is believed that siRNA 143 targets the 5′LTR HIV-1 promoter region, and suppresses HIV-1 replication in a cell infected with HIV-1. It is also believed that siRNA 136 and siRNA 205 also target the 5′LTR HIV-1 promoter region, and suppress HIV-1 replication in a cell infected with HIV-1.

1. (canceled)

2. (canceled)

3. (canceled)

4. (canceled)

5. (canceled)

6. (canceled)

7. A method of treating, preventing, or reducing an HIV infection in a subject, comprising administering to the subject an effective amount of at least one silencing nucleic acid to inhibit HIV gene transcription, wherein the at least one silencing nucleic acid is selected from the group consisting of (i) a silencing nucleic acid which targets the sequence of SEQ ID NO: 1, (ii) a silencing nucleic acid which targets the sequence of SEQ ID NO: 9, (iii) a silencing nucleic acid which targets the sequence of SEQ ID NO: 17; (iv) a silencing nucleic acid which targets a sequence having at least 95% identity with the sequence of SEQ ID NO: 1; (v) a silencing nucleic acid which targets a sequence having at least 95% identify with the sequence of SEQ ID NO: 9; and (vi) a silencing nucleic acid which targets a sequence having at least 95% identify with the sequence of SEQ ID NO: 17.

8. (canceled)

9. (canceled)

10. (canceled)

11. The method of claim 7, wherein the at least one silencing nucleic acid is an RNA duplex comprising a sense strand and an antisense strand, wherein the sense strand comprises a sequence having at least 95% identity to one of the sequence of SEQ ID NO: 6, the sequence of SEQ ID NO: 14, or the sequence of SEQ ID NO: 22.

12. (canceled)

13. The method of claim 7, wherein the at least one silencing nucleic acid is an siRNA selected from the group consisting of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; and (iii) an siRNA comprising a sense strand having the sequence of SEQ ID NO: 22, and an antisense strand having the sequence of SEQ ID NO: 23.

14. The method of claim 7, wherein the at least one silencing nucleic acid is an shRNA selected from the group consisting of (i) an shRNA having the sequence of SEQ ID NO: 8; (ii) an shRNA having the sequence of SEQ ID NO: 16; and (iii) an shRNA having the sequence of SEQ ID NO: 24.

15. A silencing nucleic acid comprising a sequence selected from the group consisting of (i) a sequence which targets a sequence in the region from about position 143 to about position 161 of the 5′ LTR of HIV-1; (ii) a sequence which targets a sequence in the region from about position 136 to about position 154 of the 5′ LTR of HIV-1; and (iii) a sequence which targets a sequence in the region from about position 205 to about position 223 of the 5′ LTR of HIV-1

16. The silencing nucleic acid of claim 15, wherein the silencing nucleic acid targets a sequence having at least 95% identity with a sequence selected from the group consisting of (i) SEQ ID NO: 1, (ii) SEQ ID NO: 9, and (iii) SEQ ID NO: 17.

17. (canceled)

18. The silencing nucleic acid of claim 15, wherein the silencing nucleic acid is an RNA duplex comprising a sense and an antisense strand, wherein the sense strand comprises a sequence having at least 95% identity to the sequence of SEQ ID NO: 6, and the antisense strand comprises a sequence that is complementary to the sense strand.

19. (canceled)

20. The silencing nucleic acid of claim 15, wherein the silencing nucleic is a siRNA comprising a sense strand having the sequence of SEQ ID NO: 6, and an antisense strain having the sequence of SEQ ID NO: 7.

21. (canceled)

22. The silencing nucleic acid of claim 15, wherein the silencing nucleic acid is a shRNA comprising the sequence of SEQ ID NO: 8.

23. (canceled)

24. (canceled)

25. (canceled)

26. (canceled)

27. (canceled)

28. The silencing nucleic acid of claim 15, wherein the silencing nucleic acid is an RNA duplex comprising a sense and an antisense strand, wherein the sense strand comprises a sequence having at least 95% identity to the sequence of SEQ ID NO: 14, and the antisense strand comprises a sequence that is complementary to the sense strand.

29. (canceled)

30. The silencing nucleic acid of claim 15, wherein the silencing nucleic acid is a siRNA comprising a sense strand having the sequence of SEQ ID NO: 14, and an antisense strain having the sequence of SEQ ID NO: 15.

31. (canceled)

32. The silencing nucleic acid of claim 15, wherein the silencing nucleic acid is a shRNA comprising the sequence of SEQ ID NO: 16.

33. (canceled)

34. (canceled)

35. (canceled)

36. (canceled)

37. (canceled)

38. The silencing nucleic acid of claim 15, wherein the silencing nucleic acid is an RNA duplex comprising a sense and an antisense strand, wherein the sense strand comprises a sequence having at least 95% identity to SEQ ID NO: 22, and the antisense strand comprises a sequence that is complementary to the sense strand.

39. (canceled)

40. The silencing nucleic acid of claim 15, wherein the silencing nucleic acid is a siRNA comprising a sense strand having the sequence of SEQ ID NO: 22, and an antisense strain having the sequence of SEQ ID NO: 23.

41. (canceled)

42. The silencing nucleic acid of claim 15, wherein the silencing nucleic acid is a shRNA comprising the sequence of SEQ ID NO: 24.

43. (canceled)

44. (canceled)

45. A composition comprising the silencing nucleic acids of claim 15 and a pharmaceutically acceptable carrier.

46. (canceled)

47. (canceled)

48. (canceled)

49. (canceled)

50. (canceled)

51. (canceled)

52. (canceled)

53. A cell comprising the silencing nucleic acid of claim 15.

54. (canceled)

55. (canceled)

56. A composition comprising at least two nucleic acid sequences, wherein the composition is selected from the group consisting of (A) at least two of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; and (iii) an siRNA comprising a sense strand having the sequence of SEQ ID NO: 22, and an antisense strand having the sequence of SEQ ID NO: 23; and (B) at least two of (i) an shRNA having the sequence of SEQ ID NO: 8; (ii) an shRNA having the sequence of SEQ ID NO: 16; and (iii) an shRNA having the sequence of SEQ ID NO: 24.

57. (canceled)

58. (canceled)

59. (canceled)

60. (canceled)

61. The composition of claim 56, wherein the composition comprises at least two of (i) an shRNA having the sequence of SEQ ID NO: 8; (ii) an shRNA having the sequence of SEQ ID NO: 16; and (iii) an shRNA having the sequence of SEQ ID NO: 24.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. The composition of claim 55, wherein the composition comprises at least two of (i) an siRNA comprising a sense strand having the sequence of SEQ ID NO: 6, and an antisense strand having the sequence of SEQ ID NO: 7; (ii) an siRNA comprising a sense strand having the sequence of SEQ ID NO: 14, and an antisense strand having the sequence of SEQ ID NO: 15; and (iii) an siRNA comprising a sense strand having the sequence of SEQ ID NO: 22, and an antisense strand having the sequence of SEQ ID NO: 23.
